(19) United States
(12) Patent Application Publication
(10) Pub. No.: US 2017/0151061 A1
(43) Pub. Date: Jun. 1, 2017

(54) Arthrodesis Implant and System Therefor

(71) Applicant: Vilex in Tennessee, Inc., McMinnville, TN (US)

(72) Inventor: Abraham Lavi, Delray Beach, FL (US)

(73) Assignee: Vilex in Tennessee, Inc., McMinnville, TN (US)

(21) Appl. No.: 15/260,960

(22) Filed: Sep. 9, 2016

Related U.S. Application Data
(60) Provisional application No. 62/216,590, filed on Sep. 10, 2015.

(51) Int. Cl. A61F 2/42

(52) U.S. Cl. CPC A61F 2/4225; A61F 2002/4228; A61F 2002/30622

(57) ABSTRACT

The invention is a compression device for arthrodesis, wherein a straight or angled bone implant device has two shafts thereon making up one compression device. The two shafts are either co-linear or one shaft is angled at a definite angle (between 0 and 15 degrees) compared to the linear angle of the other shaft. One end of the compression screw bears barbs formed by discontinuous threading, whereas the other end of the compression screw bears continuous threads or barbs formed by discontinuous threading.

### CROSS-REFERENCE TO RELATED APPLICATIONS

This patent application claims priority to, and incorporates by reference in its entirety, U.S. Patent Application No. 62/216,590 filed 10 Sep. 2015.

### BACKGROUND OF THE INVENTION

Field of the Invention

The present invention is useful in the correction of hammer/mallet/claw toe defects in the feet and comparable deformities of the hands, and by logical extension any compression arthrodesis indication.

Description of Related Art

A “hammer toe”, “mallet toe”, or “claw toe” is a toe that ordinarily remains in a flexed or curled-up orientation, and a foot can have one or multiple of these deformities. The causes of hammer/mallet/claw toes include tight shoes, arthritis, muscular or musculoskeletal imbalances and potentially other unknown etiologies.

Traditional surgeries to correct the hammer/mallet/claw toe condition have included surgical excision of bone or bone portions, insertion of complicated implants, or cutting or transplantation of associated tendons. Depending on the choice of procedure or implant in previous surgical interventions, typical complications often included one or more of nerve damage, excessive stiffness, or even excessive blood loss or infection.

A need therefore remains for a hammer/mallet/claw toe implant for which surgical insertion is easy, blood, bone and tendon loss are all minimal, and for which patient recovery is straightforward and as painless as possible.

### SUMMARY OF THE INVENTION

In order to meet this need, the invention is a uniquely configured, typically cannulated, implant well suited for arthrodesis of the proximal phalanx and the intermediate phalanx of the human toe—or comparable arthrodesis indications, including deformities of the hand analogous to hammer toe conditions of the foot. The implant is a single shaft, usually made of surgical grade titanium or another appropriate metal but feasibly of any material suitable for bone implantation. The shaft typically has a proximal portion and a distal portion. The distal portion bears a segment of deep, interrupted threads that resemble barbs, with the barbs' typically being positioned at 120 or 180 degrees around the axis of the implant, whereas the proximal portion is threaded with generally continuous threads of a typically shallower dimension than the distal deep, interrupted threads. The barbed end is designed to rotate, reach into and engage with the inner cortex of the intramedullary canal of the intermediate phalanx (or other applicable bone or bone segment) and rotate back and pull the intermediate phalanx back toward the proximal phalanx while compressing the bones into position upon final positioning of the implant. A first embodiment of the invention has collinear proximal and distal portions of the shaft of the implant; a second embodiment has a distal (barbed end) portion which is angled 10 degrees from linear with respect to the proximal portion of the shaft of the implant. For podiatric applications, a smaller version is intended for use in mallet toe, namely, to bring together the distal and intermediate phalanges (or comparable other arthrodesis targets).

### BRIEF DESCRIPTION OF THE DRAWINGS

FIG. 1a is distal end perspective view of a first embodiment of the present invention;

FIG. 1b is a perspective view of the inventive embodiment of FIG. 1a;

FIG. 1c is a further perspective view of the inventive embodiment of FIG. 1a;

FIG. 2a is a perspective view of a second embodiment of the invention;

FIG. 2b is a perspective view of a third embodiment of the invention;

FIG. 3a is a perspective view of a fourth embodiment of the invention;

FIG. 3b is a perspective view of the inventive embodiment of FIG. 3a shown with a guide wire in place in the cannulation of the inventive implant;

FIG. 3c is a perspective view of the fourth embodiment of the invention; and

FIG. 4 is a side sectional view of the second embodiment of the invention.

### DETAILED DESCRIPTION OF THE INVENTION

As described above, the invention is an implant well suited for arthrodesis of the proximal phalanx and the intermediate phalanx of the human toe or other bones requiring arthrodesis. The implant is a single shaft made of suitable bone implant material. The shaft generally has a proximal portion and a distal portion. The distal portion bears a segment of deep, interrupted threads that resemble barbs, with the barbs' typically being positioned at 120 degrees around the axis of the implant, whereas the proximal portion is threaded with generally continuous threads of a typically relatively shallower dimension. The barbed end is designed to rotate, reach into and engage with the inner cortex of a first bone and rotate back and pull the first bone back toward the second bone while compressing the bones into final position. One embodiment of the invention has co-linear proximal and distal portions of the shaft of the implant; another embodiment has a distal (barbed end) portion which is angled 10 degrees from linear with respect to the proximal portion of the shaft of the implant. For podiatric applications, a smaller version is intended for use in a 180-degree opposite orientation, namely, to bring together the distal and intermediate phalanges (or comparable other arthrodesis targets). In yet another embodiment of the invention, the shaft diameters of the proximal and distal portions of the implant are generally similar, with the distal discontinuous threads' having a diameter no more than 20% larger than, and more preferably no more than 10% larger than, the diameter of the proximal continuous threads. These and other embodiments of the invention are discussed later in this patent specification. For ease of reference, compression screws with ten degree angled shafts are sometimes referred to as “ten degree implants” and compression screws without the angled shaft are sometimes referred to as “zero degree implants.”

The implants of the present invention can be constructed with or without cannulation. Cannulation does not form a key feature of the present invention. Cannulation can make placement with a guide wire advantageous but the invention can be practiced with non-cannulated implants also. Just as cannulation itself is not critical to the concept of the present invention, cannulation diameter is likely not critical as long as any cannulation present is capable of admitting a guide wire of tenable dimension, that is, not too thin or fine to be practical.

In the above description of the embodiments of the invention generally, reference is made to distal shafts' having discontinuous threads (and barbs) with the proximal shafts' being continuously threaded. For hammer toe correction and concomitant arthrodesis of the intermediate phalanx of the toe with the proximal phalanx of the same toe, the convention of the implant's distal shaft's bearing the barbs and the proximal shaft's bearing the continuous threads would apply. However, because the present implant can be used in any compression arthrodesis indication, sometimes the barbs are on the proximal shaft and the continuous threads are on the distal shaft. Accordingly, in the below description of the Figures, instead of referring to the proximal and distal shafts of the present compression screw, the naming convention refers to the first shaft and second shaft, which depending on the specific indication may be the proximal and distal shafts of the implant, respectively, or might otherwise be the distal and proximal shafts of the invention, respectively. Typically, but by no means always, the bone or bone segment that will be affixed to the discontinuous, barbed shaft of the present implant will be the bone or bone segment that is most at risk. So, for example, in a hammer toe correction the intermediate phalanx is the bone most at risk and the intermediate phalanx thus is the recipient of the discontinuously threaded, barbed shaft of the implant during hammer toe correction. Those skilled in the art will therefore appreciate that in the ensuing description the first shaft of the present implant will generally, but by no means always, be the distal shaft of the implant and the second shaft of the present implant will generally, but by no means always, be the proximal shaft of the implant.

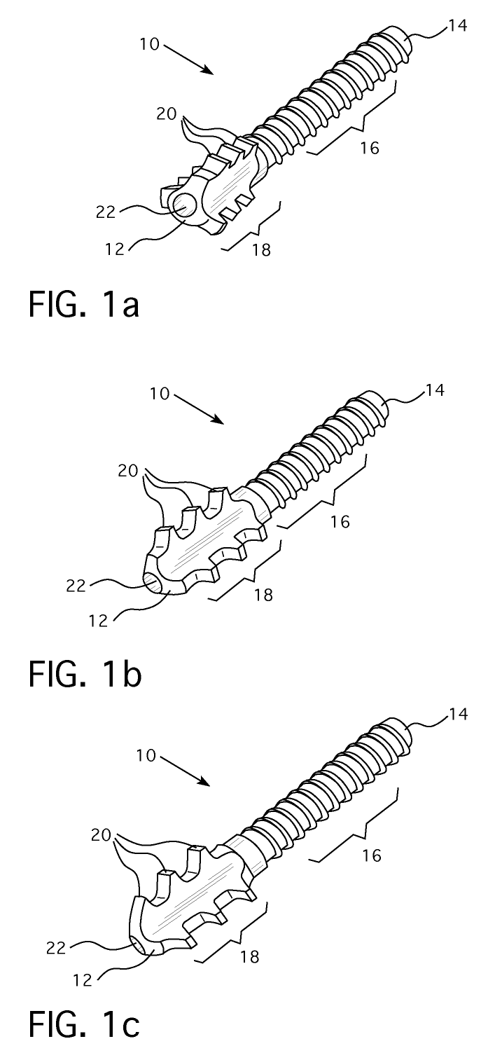

Referring now to FIG. 1a, the compression screw 10 (first embodiment) is made up of the first shaft 12 and the second shaft 14. The first shaft 12 has a series of interrupted screw threads thereon, or interrupted threads 18, whereas the second shaft 14 has continuous threads 16 thereon. The interrupted threads 18 of FIG. 1a create three lines of barbs 20, 120 degrees apart on the axis of the compression screw 10. An optional axial cannulation 22 is shown in the perspective view of FIG. 1a.

Referring now to FIG. 1b, the same compression screw as shown in FIG. 1a is shown in a self-evidently axially rotated orientation. As in FIG. 1a, the compression screw of FIG. 1b has first and second shafts 12, 14 making up the compression screw 10, with interrupted threads 18, continuous threads 16, and barbs 20, along with a cannulation 12. As with all embodiments of the present invention, the cannulation is optional.

FIG. 1c is a perspective view of the same embodiment of the invention shown in FIGS. 1a and 1b, but more from the side than from the end to give a more direct illustration of the spacing between the barbs 20. The other components of the compression screw 10 of FIG. 1c are the same as in FIG. 1a and FIG. 1b.

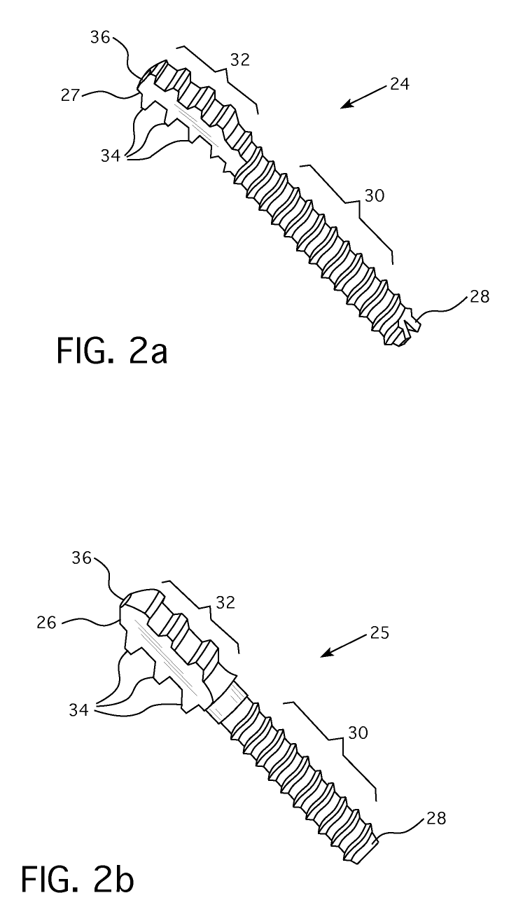

Referring now to FIG. 2a, a second embodiment of the invention is shown in which the first shaft 27 of the compression screw 24 is angled 10 degrees off-axis relative to the axis of the second shaft 28 of the compression screw 24. Also, the first shaft 27 has two lines of barbs 34 positioned 180 degrees relative to the axis of the compression screw 24. The compression screw 24 also has continuous threads 30, interrupted threads 32 (the interrupted threads 32 as a whole make up the individual barbs 34) and a cannulation 36. Note that the cannulation 36 is not strictly speaking an axial cannulation in the sense that there is no straight-line axis to the compression screw 24 of the second embodiment of the invention.

Referring now to FIG. 2b, a third embodiment of the invention is the same as the second embodiment shown in FIG. 2a except that the first shaft 26 of compression screw 25 is in-line with the second shaft 28. Otherwise the features of the compression screw 25 are the same, with interrupted threads 32 creating individual barbs 34, continuous threads 30 and a cannulation 36—in this case an axial cannulation because the entire compression screw 24 is in-line. As with every embodiment of the invention, even though the cannulation of FIGS. 2a and 2b are shown in the drawings, in fact cannulations are always optional according to the present invention.

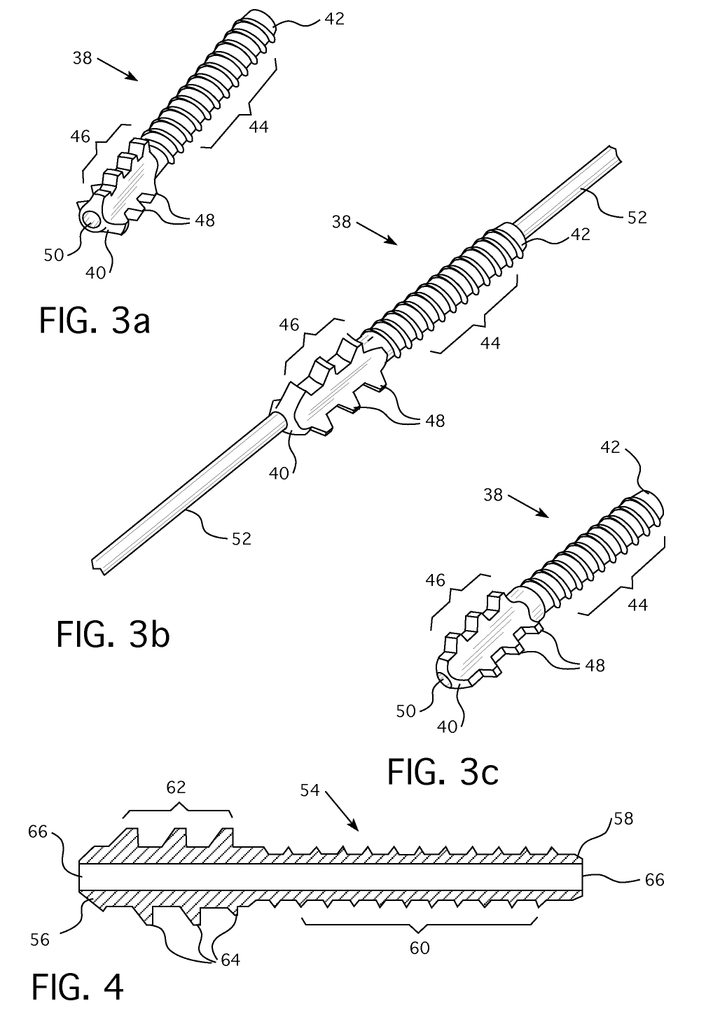

Referring now to FIG. 3a, the compression screw 38 has a first shaft 40 and a second shaft 42 and represents a fourth embodiment of the present invention. The first shaft 40 bears discontinuous thread 46 which create the individual barbs 48 as shown. The second shaft 42 has continuous threads 44 thereon. The lines of barbs 48 are 120 degrees apart relative to the axis of the compression screw 38. A distinctive feature of the compression screw 38 of FIG. 3a is the relative diameters of the first shaft 40 and the second shaft 42—these two shafts are in general of the same or comparable diameter not counting the protrusion of the threads. According to the fourth embodiment of the invention, the minor (threadless) diameters of the first and second shafts 40, 42 are within 20% of one another, preferably within 10% of one another. In general, the fourth embodiment of the present invention is best suited for the smallest gauges of compression screws for the smallest diameter compression arthrodeses.

Referring now to FIG. 3b, the same fourth embodiment of the invention as shown in FIG. 3a appears in perspective view, with all the same components as shown, but with the guide wire 52 in place within the axial cannulation 50. FIG. 3c illustrates the same fourth embodiment in a relatively rotated perspective view, as contrasted with FIG. 3a, but with no other difference.

Finally, FIG. 4 is a side sectional view of the third embodiment of the invention as shown in FIG. 2b. The compression screw 54 has a first shaft 56, a second shaft 58, interrupted threads 62 which create individual barbs 64, continuous threads 60 and an axial cannulation 66.

The surgical technique for implanting the present compression screw can include the following, styled for exemplary purposes as a hammer toe repair of the proximal and intermediate phalanges of a toe of a human foot. First, the surgeon locates the dorsal surface of the proximal-intermediate-phalangeal (PIP) joint and creates an incision centrally on this dorsal surface, with resection of the soft tissue around the joint to expose the bone segments. The proximal phalanx is the resected based on the desired post-surgical joint angle of either zero degrees or ten degrees, corresponding to the implant selected. In other words, a resection perpendicular to the medullary canal of the proximal phalanx will correspond to a zero degree implant, whereas a resection angled 10 degrees plantar from the medullary canal for a plantar-biased position will correspond to a ten degree implant.

After the above-described soft tissue and bone resections, typically a guide wire is inserted into the medullary canal of the intermediate phalanx. The guide wires are sized to accompany the compression screw size indicated in the particular arthrodesis—matching guide wires to implant cannulations is well known in the art. Optimally, bi-planar fluoroscopy (medial/lateral and dorsal/plantar) is used to ensure that the guide wire is located centrally within the canal of the proximal phalanx. If necessary, wire repositioning should recur until a satisfactory position is achieved.

Preferably a cannulated stop drill is advanced over the guide wire to drill into the medullary bone of the intermediate phalanx. The stop drill is dimensioned to correlate to the dimension of the inventive compression screw according to the skill of the art. A broach can be advantageously used to assure correct rotational alignment of the drilled passage. After drilling and broaching, the broach and guide wire should be removed from the intermediate phalanx.

Typically in a PIP hammer toe correction, the implant itself will be lodged in the proximal phalanx first, after preparation of the intermediate phalanx as described above. The guide wire is typically inserted into the medullary canal of the proximal phalanx generally as it was placed in the intermediate phalanx. Then, the selected compression screw is loaded onto an appropriate driver instrument which, if applicable, is configured to accommodate the 10 degree-off-axis angle of the compression screw (second embodiment of the invention). The continuous screw shaft of the implant is then inserted into the proximal phalanx medullary bone—if a guide wire is in place, then a cannulated implant is inserted continuous-thread-end first into the proximal phalanx medullary bone—and also the driver tool is removed.

After all the above steps have been completed, the PIP joint is ready for manual compression screw arthrodesis as follows. With the continuous-thread end of the compression screw affixed in the medullary bone of the proximal phalanx, the surgeon then presses the barbed region (discontinuous thread implant shaft) into the broached hole in the intermediate phalanx, ensuring the two phalanges are aligned. While it is possible simply to pressure-fit the barbed region into the intermediate phalanx as described above, the present compression screw's discontinuous thread design is uniquely suited to a compression-twisting installation in which the surgeon gently rotates the intermediate phalanx by about 20-45 degrees, presses the barbed end of the compression screw into the intermediate phalanx with finger pressure, and gently re-rotates the intermediate phalanx back to facilitate the seating of the barbs in the medullary bone of the intermediate phalanx. After full seating of the compression screw in the respective bone segments, no gap will persist and compression arthrodesis is complete. Preferably, after installation of the present compression screw bi-planar fluoroscopy (see above) is used to verify that the implant is fully seated within each bone segment and that the two phalanges are in bone-to-bone contact across the resected regions.

The above-described surgical procedures is exemplary only. Because the compression screw of the present invention can be used in any compression arthrodesis indication, whether the barbed shaft is distal or proximal is a matter of surgeon's preference. As reported above, generally but not always the barbed shaft will be in the bone or bone segment most at risk—and since the most distal bone segment is generally the most at risk, the barbed shaft will generally (but not always) be the distal shaft of the present compression screw. One particular advantage of the barbs is that the barbs tend to seat very firmly into their respective medullary bone and even lodge more securely if the barbs are rotated into place to anchor them in the bone. The inventors therefore regard as the key to their invention the double conjunction of barbs (composed of interrupted, discontinuous threading) at one end and continuous threads at another

Although the present implant can be made of any suitable material, surgical stainless steel, cobalt chrome or titanium alloys are preferred.

Although the present invention has been described with particularity above, with specific mention of components, configurations, relative dimensions and references to degrees of position relative to implant axes, the invention is only to be limited insofar as is set forth in the accompanying claims.

The invention claimed is:

1. A compression device for arthrodesis, comprising a one piece implant having a first shaft and a second shaft thereon, wherein said first shaft bears discontinuous threads which create barbs thereon, and said second shaft bears continuous threads thereon or discontinuous threads which create barbs thereon.

2. The compression device according to claim 1, wherein said first shaft bears barbs in three lines generally parallel to the axis of the compression device and spaced 120 degrees apart around the circumference of the compression device relative to said axis.

3. The compression device according to claim 1, wherein said first shaft bears barbs in two lines generally parallel to the axis of the compression device and spaced 180 degrees apart around the circumference of the compression device relative to said axis.

4. The compression device according to claim 1 wherein said first shaft is angled 10 degrees from the axial angle of said second shaft.

5. The compression device according to claim 1 wherein the compression device is cannulated.

6. The compression device according to claim 5 wherein said compression device is dimensioned to admit a guide wire in the cannulation.

7. The compression device according to claim 1 wherein said barbs have a pitch greater than the pitch of said continuous threads.

8. The compression device according to claim 1 wherein said barbs have a diameter greater than the diameter of said continuous threads.

9. The compression device according to claim 1 wherein said barbs have a pitch lesser than the pitch of said continuous threads.

10. The compression device according to claim 1 wherein said barbs have a diameter less than the diameter of said continuous threads.
